(19) United States
(12) Patent Application Publication
(10) Pub. No.: US 2004/0260284 A1
(43) Pub. Date: Dec. 23, 2004

(54) Anti-splay pedicle screw

(76) Inventor: Matthew Parker, Houston, TX (US)

Correspondence Address: AKIN, GUMP, STRAUSS, HAUER & FELD, 1111 LOUISIANA STREET, 44TH FLOOR, HOUSTON, TX, 77002, US

(21) Appl. No.: 10/601,695

(22) Filed: Jun. 23, 2003

(51) Int. Cl. A61B 17/56

(52) U.S. Cl. 606/61

(57) ABSTRACT

A pedicle screw provides for elimination of splaying forces caused by an internal closure member. A locking member dovetails with longitudinal channels in wall sections of the receiver, locking the wall sections together and preventing the splaying. The closure member when disposed within a bore of the receiver tightens the locking member onto an elongated member, preventing removal of the locking member and the elongated member from the receiver. The locking member and the closure member may be permanently rotatably inter-engaged for ease of insertion and removal.

### BACKGROUND OF THE INVENTION

1. Field of the Invention

The present invention relates to medical devices and in particular to a pedicle screw.

2. Description of the Related Art

A pedicle screw is a form of specialized screw used in surgery for the stabilization and immobilization of spinal segments to relieve instability for trauma purposes, and for the relief of pain due to degenerative disc disease. The screw is composed of multiple components generally including a screw shank, a receiver or screw head, a closure cap, and a rod which is used to connect two or more screws together in a series. The screw shank typically extends through the bottom of the screw head and is held in place in a polyaxial joint that allows for flexibility to adapt to the desired position of the vertebra. The closure cap is then typically inserted down on top of the rod and threaded into the receiver or head, locking the rod down tightly to the receiver as well as locking down the polyaxial joint between the receiver and screw shank.

There are several different types of pedicle screws with various closure mechanisms currently available. Internal closure caps offer advantages that have made internal closure caps widely used in pedicle screws. One such advantage is the ease of use. Internal closure caps provide a clean, visible, protected spot to place the closure cap. However, internal closure caps inherently act as a wedge, splaying or spreading the “horseshoe” shape of the receiver as the cap tightens down on the rod, or afterwards, tending to splay in response to stress caused by patient movement. This splaying can lead to a compromise of the connection between the closure cap and the receiver. This is a known problem. There have been documented instances where closure caps have popped out of or dislocated within receivers causing immediate and catastrophic failure of the pedicle screw system. A number of patents are directed to solving this problem, but the search for a better solution continues.

Because of the splaying problem inherent to an internal closure cap, some pedicle screws have utilized an outer nut to avoid the splaying problem. However, an outer nut leads to its own set of problems. The outer nut tends to bind in soft tissue that surrounds the vertebra which can cause complications and increase the difficulty in attempting to attach the nut. Outer nuts can also lead to increased difficulty and application of other instruments that commonly used to perform correction maneuvers. These instruments are often used in other parts of the surgery and are often used in conjunction with tightening and loosening of the closure caps.

Pedicle hooks, similar to pedicle screws but using a hook mechanism instead of a screw shank, are also used in certain surgical situations. The hook is typically formed as a single unit with the receiver.

### BRIEF SUMMARY OF THE INVENTION

Various embodiments implementing aspects of the invention are disclosed, generally directed to an internally closed receiver with a locking member to prevent splaying of the receiver. The locking member locks into a longitudinal channel in the walls of the receiver. In one embodiment, the locking member and a closure member are rotatably attached to each other for insertion into the receiver. In various embodiments, the receiver is attached to the screw shank for universal movement, the shank can be affixed to the receiver, or the receiver is attached to a hook for attachment to a bone.

### BRIEF DESCRIPTION OF THE SEVERAL VIEWS OF THE DRAWINGS

A better understanding can be obtained when the following detailed description of several disclosed embodiments is considered in conjunction with the following drawings in which

FIG. 1 is a cutaway view of a pedicle screw according to one embodiment;

FIG. 2 is a cutaway view of a portion of the embodiment of FIG. 1 from an angle perpendicular to the view of FIG. 1;

FIG. 3 is a top view of a locking member engaged with the screw head of the pedicle screw according to one embodiment;

FIG. 4 is a top view of the embodiment of FIG. 3 showing a closure member holding the locking member in place;

FIG. 5 is an exploded view of the screw head locking member and closure member according to one embodiment;

FIG. 6 is a view of three pedicle screws connected with a rod into a single unit;

FIG. 7a is a cutaway view of a pedicle hook according to one embodiment; and

FIG. 7b is a view of the pedicle hook of FIG. 7a from a different angle.

### DETAILED DESCRIPTION OF THE INVENTION

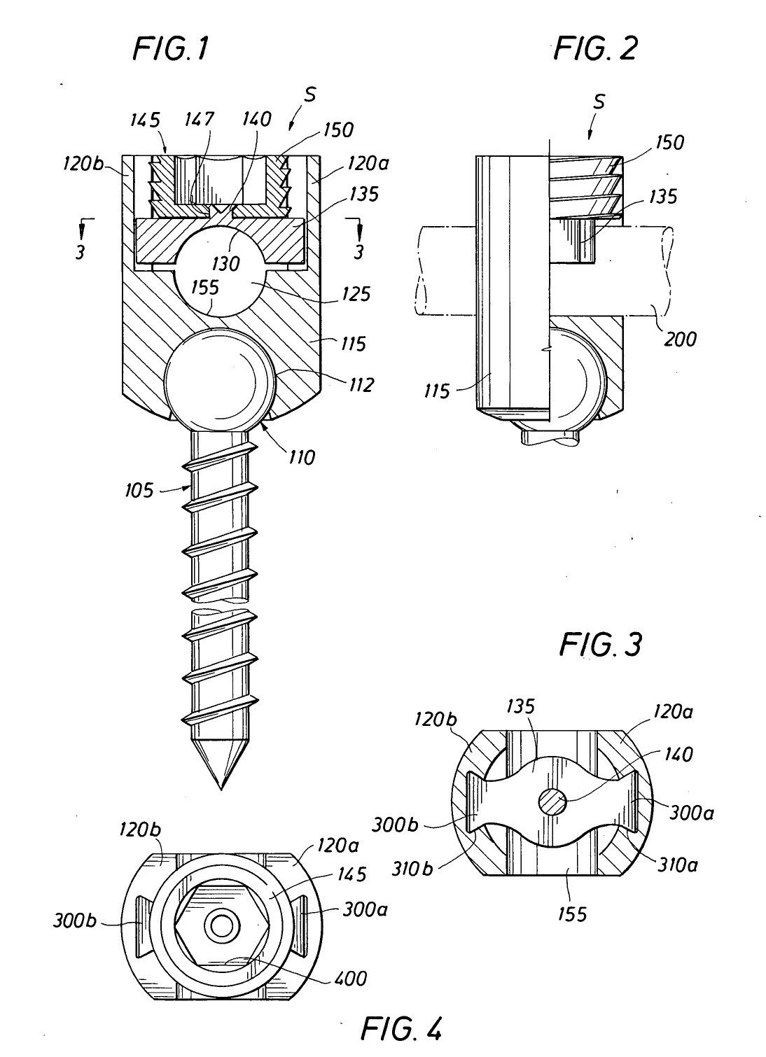

A pedicle screw of an internal plug design with an additional locking member actively resists the aforementioned splaying or spreading forces. Disclosed embodiments provide for easy insertion of the closure cap and prevent any compromise of strength due to splaying of the screw head. FIG. 1 is a cutaway view of a pedicle screw S according to one embodiment assembled together. The screw shank 105 is a threaded portion of a screw that is driven or otherwise inserted into the pedicle of a vertebra to provide an anchor point. Screw shanks 105 come in a variety of outer thread diameters, typically between 4.5 mm and 8.5 mm. The length of the screw shank 105 is determined by measuring from right below the head of the screw shank 105 to the tip of the screw shank 105. The head of the screw shank 105 is not typically included in the length measurement. Lengths typically range between 25 mm and 60 mm. A thread pitch of the screw shank 105 can vary. In one embodiment, the head 110 of the screw shank 105 is spherical in nature. A drive slot (not shown) may be milled into end of the head 110 in line with the midline of the screw shank 105 in order to drive the screw shank 105 into and out of the bone of the vertebra. Other techniques for driving the screw shank 105 into the bone can be used.

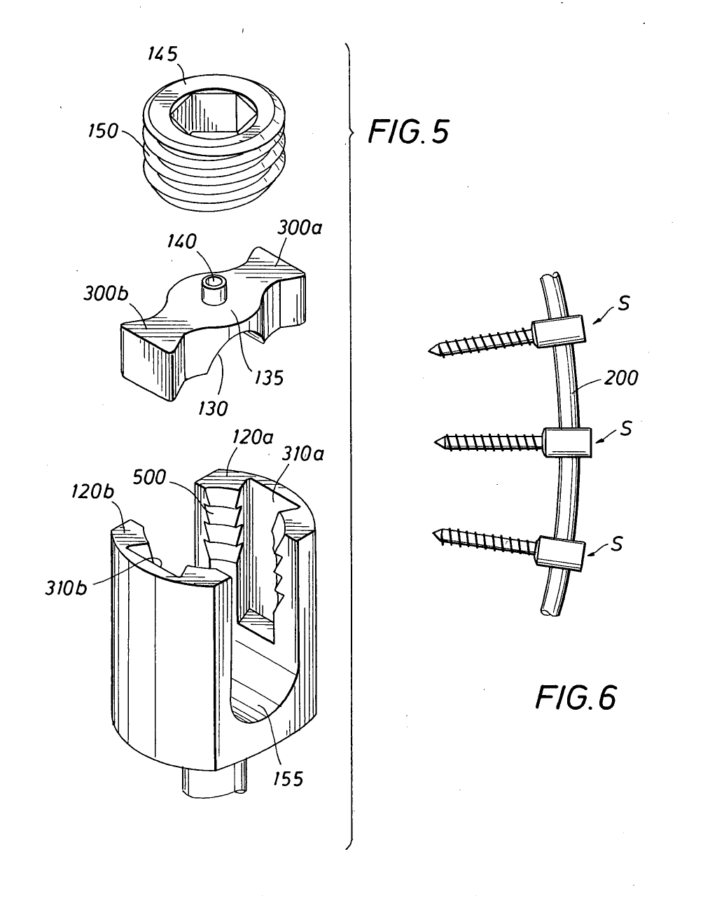

A receiver or head 115 as in a disclosed embodiment may perform multiple functions. The polyaxial head 115 includes a floating saddle 112 where the polyaxial screw head 110 articulates, giving the polyaxial screw shank 105 polyaxial capability. The receiver 115 also acts as a receiver for a rod (not shown in FIG. 1) for connecting multiple screws S to each other. The receiver 115 forms a female portion or recess to receive the closure cap 145 and locks the entire construct down. The receiver 115 typically is generally shaped as a cylinder between 13 mm and 16 mm in diameter and between 15 mm and 17 mm in length. The receiver 115 may contain features to allow for mechanical assistance in seating the rod into the receiver 115. These features are well known in the art and will not be further discussed. A bore is formed into the upper portion of the receiver 115. A transverse channel 155 is also formed perpendicular to the cylindrical axis of the receiver 115 for accepting the rod that connects multiple screws S together. Rods may have different outer diameters, with a 5.5 mm rod being typical. As best shown in FIG. 5, the receiver 115 thus contains a plurality of walls 120A and 120B surrounding the transverse channel 155. Typically the upper end of the inside surface of the plurality of walls 120A and 120B will have threads 500 for screwing an internal closure member into the polyaxial head 115. As best shown in FIG. 5, each of the plurality of walls 120A and 120B contains a longitudinal channel 310A and 310B. This channel is typically perpendicular to the transverse channel for the rod and typically extends into the threaded portion 500 of the walls 120. However, other channel orientations may be used, such as spiral orientation.

A locking member 135 can be inserted into the receiver 115. The lower surface of the locking member 135 may contain a channel 130 for receiving the rod and holding the rod in place. The channel 130 typically mirrors the channel 155 of the receiver 115. In one embodiment, a pin or other protrusion may extend perpendicular to an upper surface of the locking member 135 for engagement with the closure member 145. As shown in FIG. 1, the pin 140 can be splayed outwardly after engagement with the closure member 145 to allow the closure member 145 and the locking member 135 to be held together for ease of use in surgery, while allowing rotation of the two pieces with respect to each other. The closure member 145 may contain threads 150 to mate with the threaded portion 500 of the plurality of walls 120 for securely tightening the locking member 135 onto the rod 200. Other techniques for mating the closure member 145 with the walls 120 can be used.

As shown in FIG. 2, when the locking member 135 is placed into the receiver 115, it can saddle the rod 200 in the channel 130, thus allowing fixing the rod 200 in place.

As shown in the top view of FIG. 3, tenons 300a and 300b of the locking member 135 are mortised into longitudinal channels 300a and 300b upon insertion of the locking member 135. The locking member 135 is thus dovetailed into the plurality of walls 120a and 120b, preventing splaying of the walls 120a and 120b, when the closure member 145 is threaded into or otherwise inserted into the receiver 115, as shown in the top view of FIG. 4. As shown in the top view of FIG. 4, the closure member 145 may have a hexagonal shaped opening for engaging with an insertion tool. Although as shown in FIG. 4, the opening is hexagonal in shape, other shapes may be used, corresponding to the insertion tool (not shown). Although as shown, the channels 300a and 300b are roughly triangular in cross-section, other cross-sectioned shapes in which the width of the channel 300 distal from the bore in the receiver 115 is larger than the width of the channel 300 proximal to the bore may be used. For example, a T-shaped channel may be used.

As best shown in FIG. 5, the locking member 135 may have a vertical pin 140 for insertion into an opening 147 of the closure member 145. In another embodiment, the locking member 135 is not attached to the closure member 145 but can be separately inserted into the receiver 115. Other forms of attachment allowing rotation between the locking member 135 and the closure member 145 may be used.

FIG. 6 illustrates the use of pedicle screws S with a common rod 200 locking multiple vertebra in a desired alignment.

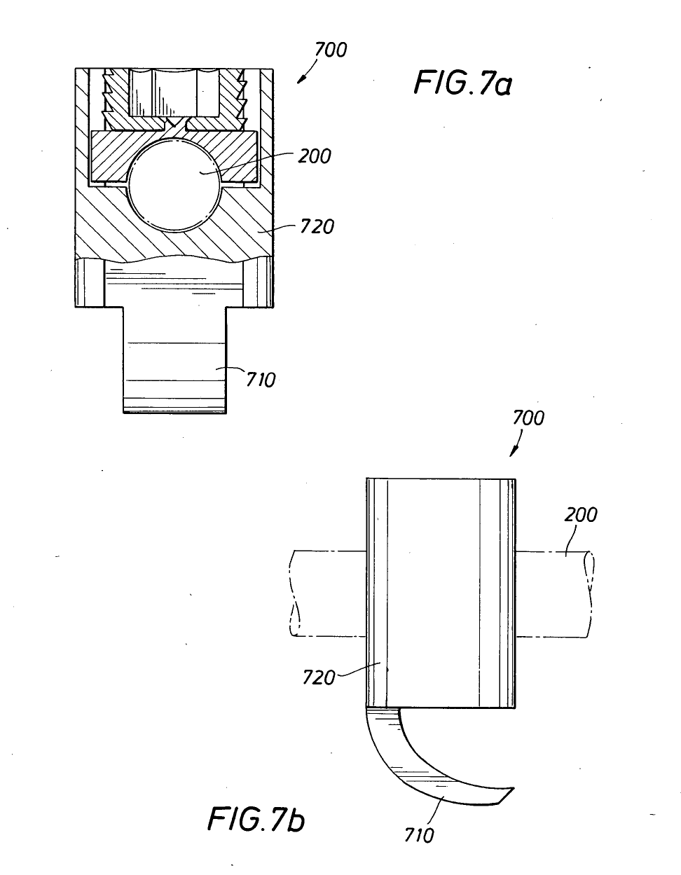

In some situations, a pedicle hook 700 as shown in FIGS. 7a-7b may be preferable to use of a pedicle screw S as discussed above. FIG. 7a is a cutaway view of the pedicle hook 700 from an angle parallel to the rod 200, shown in phantom. FIG. 7b is a view of the pedicle hook 700 from an angle perpendicular to the 200. The pedicle hook 700 is typically a unitary receiver 720 as in the pedicle screw S, substituting a hook 710 for the screw shank 105 and floating saddle 112. However, other techniques for attaching the hook 710 to the receiver 720 can be used. The shape, size, and orientation of the hook 710 shown in FIGS. 7a-7b is exemplary and illustrative only, and other shapes, sizes, and orientations can be used. Both open and closed hooks are known in the art, with closed hooks typically open for insertion and closed with a surgical tool upon proper placement. The receiver 720 of the pedicle hook in a disclosed embodiment is otherwise identical to the receiver 115 of the pedicle screw S of FIGS. 1-6.

The foregoing disclosure and description of the various embodiments is illustrative and explanatory thereof, and various changes in the elements and details of the illustrated elements, construction and method of operation may be made without departing from the spirit of the invention.

I claim:

1. A pedicle screw system, comprising:
an attachment member; and
a receiver, disposed with the attachment member, comprising:
a plurality of wall sections defining a longitudinal bore in the receiver and a first transverse channel perpendicular to the bore, the first transverse channel adapted to engage with an elongated member, each of the plurality of wall sections comprising:
a longitudinal channel, the channel having a first width proximal to the bore and a second width distal to the bore, the second width larger than the first width;
a closure member removably disposable within the bore; and
a locking member, removably disposable within the bore between the closure member and the elongated member, the locking member shaped to interlock with the longitudinal channels,
wherein the closure member, when disposed within the bore with the locking member, prevents removal of the locking member from the receiver.

2. The pedicle screw system of claim 1, wherein the longitudinal channels have a substantially triangular cross-section.

3. The pedicle screw system of claim 1, wherein the longitudinal channels have a substantially T-shaped cross-section.

4. The pedicle screw system of claim 1, the closure member threadedly engageable with the plurality of wall sections.

5. The pedicle screw system of claim 1, wherein the locking member and the closure member are inter-engaged such that the locking member and the closure member can swivel relative to each other.

6. The pedicle screw system of claim 5, the locking member comprising:
a locking body, shaped to interlock with the longitudinal channels; and
a pin, central to and extending perpendicular to the locking body, the pin engaging with a central opening in the closure member.

7. The pedicle screw system of claim 6, wherein the pin extends through the central opening of the closure member when the closure member and the locking member are inter-engaged, the pin flared to prevent disengagement of the closure member and the locking member.

8. The pedicle screw system of claim 1, wherein the closure member is self-threading.

9. The pedicle screw system of claim 1, the locking member comprising:
a second transverse channel, the second transverse channel adapted to fix the elongated member between the first transverse channel and the second transverse channel when the locking member is disposed within the bore and tightened against the elongated member by the closure member.

10. The pedicle screw system of claim 1, wherein the attachment member is a screw shank, the screw shank removably disposed with the receiver.

11. The pedicle screw system of claim 10,
wherein the screw shank has a polyaxial head, and
wherein the polyaxial head of the screw shank is disposed within a polyaxial joint of the receiver,
whereby the screw shank can be universally positioned relative to the longitudinal axis of the receiver.

12. The pedicle screw system of claim 10, wherein the screw shank has a drive slot in a head portion of the screw shank, the drive slot adapted for insertion and positioning of the screw shank.

13. The pedicle screw system of claim 1, wherein the attachment member is a hook.

14. The pedicle screw system of claim 13, wherein the hook is integral with the receiver.

15. A method of capturing an elongated member in a receiver member of a medical device, comprising:
inserting the elongated member within a transverse channel between a plurality of walls surrounding a longitudinal bore in the receiver;
saddling the elongated member with a locking member disposed within the bore perpendicular to the elongated member;
dovetailing the locking member with a longitudinal channel formed in each of the plurality of walls, preventing outward movement of the plurality of walls;
tightening the locking member onto the elongated member.

16. The method of claim 15, tightening the locking member comprising:
rotating a closure member within the bore,
wherein rotating the closure member within the bore urges the locking member against the elongated member.

17. The method of claim 16, wherein rotating the closure member comprises:
threading the closure member in a threaded section formed around the bore in an interior surface of each of the plurality of walls.

18. The method of claim 16, further comprising:
rotatably inter-engaging the closure member and the locking member prior to insertion into the bore.

19. The method of claim 18, rotatably inter-engaging the closure member and the locking member comprising:
extending a pin from a surface of the locking member perpendicular to the locking member;
inserting the pin into an opening in the closure member;
flaring the pin, preventing removal of the locking member from the closure member.
